(19) United States
(12) Patent Application Publication
(10) Pub. No.: US 2007/0016035 A1
(43) Pub. Date: Jan. 18, 2007

(54) Ultrasonic diagnostic apparatus and ultrasonic image generating method

(75) Inventor: Hiroshi Hashimoto, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/435,025

(22) Filed: May 16, 2006

(30) Foreign Application Priority Data
May 17, 2005 (JP) 2005-143874

(51) Int. Cl. A61B 8/00

(52) U.S. Cl. 600/437

(57) ABSTRACT

With the objective of definitely generating a biopsy needle on an ultrasonic image, an ultrasonic diagnostic apparatus according to the present invention brings a probe connected thereto into contact with the surface of a body and applies ultrasound thereto, receives signals reflected from within the body and the biopsy needle inserted in the body, and generates ultrasonic images (tomograms) in the body and at the biopsy needle on the basis of the received signals. Picture elements are added every pixels with respect to range-specified portions of these ultrasonic images to thereby generate an ultrasonic image formed by superimposing the respective pixels. Even while the so-superimposed ultrasonic image is being created, ultrasonic images are sequentially stored. The so-superimposed ultrasonic image and the latest ultrasonic image are displayed on an output unit in combination.

### CROSS REFERENCE TO RELATED APPLICATIONS

This application claims the benefit of Japanese Application No. 2005-143874 filed May 17, 2005.

### BACKGROUND OF THE INVENTION

The present invention relates to an ultrasonic diagnostic apparatus, an ultrasonic image generating method and an ultrasonic image generating program wherein ultrasound is applied to within a body to be examined through an ultrasound probe, ultrasonic sounds reflected from within the body and a biopsy needle inserted in the body are received by the ultrasound probe and ultrasonic images are sequentially generated.

Paracentesis done for the purpose of tissue diagnosis and cytological diagnosis relative to a specific area in a body, drainage, implantation of radioactive materials, etc. has heretofore been carried out under images drawn by an ultrasonic diagnostic apparatus. In a patent document 1 (Japanese Unexamined Patent Publication No. Sho 61(1986)-31129), for example, an ultrasonic diagnostic apparatus is activated while a probe (ultrasound probe) is brought into contact with the surface of a body and a biopsy needle is being inserted in the body through a biopsy needle inserting attachment mounted to the probe, thereby making it possible to simultaneously confirm an image of in-vivo tissue and the movement of the biopsy needle on a display.

[Patent Document 1] Japanese Unexamined Patent Publication No. Sho 61(1986)-31129

Meanwhile, the prior art referred to above is accompanied by a problem that it is difficult to draw a biopsy needle on an ultrasonic image as will be described below. That is, the prior art has a problem in that there are many cases in which since the angle of an ultrasonic beam relative to the biopsy needle is not taken into consideration at all, the probe is not capable of receiving the ultrasonic beam from the biopsy needle, which is substantially mirror-reflected; and a signal is weak even though the probe has received it, whereby the biopsy needle is hard to be definitely displayed on the corresponding ultrasonic image (e.g., the biopsy needle is displayed by snatches).

### SUMMARY OF THE INVENTION

It is therefore an object of the present invention to provide an ultrasonic diagnostic apparatus, an ultrasonic image generating method and an ultrasonic image generating program, which can definitely display a biopsy needle on ultrasonic images.

In order to solve the problems and attain the above object, the invention of a first aspect provides an ultrasonic diagnostic apparatus which applies ultrasound to within a body to be examined through an ultrasound probe, receives ultrasonic sounds reflected from within the body and a biopsy needle inserted in the body by means of the ultrasound probe and sequentially generates ultrasonic images, comprising ultrasonic image storing means which sequentially stores the ultrasonic images therein; ultrasonic image superimposing means which sequentially superimposes the ultrasonic images stored in the ultrasonic image storing means in order of generation thereof; and superimposed-image generating means which generates an image superimposed by the ultrasonic image superimposing means.

According to the invention of the first aspect, since the ultrasonic images are generated in superimposed form, the ultrasonic diagnostic apparatus is capable of definitely displaying the biopsy needle inserted in the body, which is hard to be well displayed (in which the biopsy needle is drawn by snatches, for example).

The invention of a second aspect provides an ultrasonic diagnostic apparatus according to the above invention wherein the superimposed-image generating means superimposes images around the biopsy needle on one another.

According to the invention of the second aspect, since the images around the biopsy needle are generated in superimposed form, the ultrasonic diagnostic apparatus is capable of definitely displaying only the periphery of the biopsy needle in each ultrasonic image.

The invention of a third aspect provides an ultrasonic diagnostic apparatus according to the above invention wherein the superimposed-image generating means adds the ultrasonic images stored in the ultrasonic image storing means every pixels.

According to the invention of the third aspect, since the ultrasonic images are sequentially added every pixels, the ultrasonic diagnostic apparatus is capable of definitely displaying the biopsy needle in the ultrasonic image by a relatively simple process.

The invention of a fourth aspect provides an ultrasonic diagnostic apparatus according to the above invention wherein the superimposed-image generating means superimposes the ultrasonic images stored in the ultrasonic image storing means on one another in averaged form every pixels.

According to the invention of the fourth aspect, since the ultrasonic images are sequentially superimposed on one another in averaged form every pixels, the ultrasonic diagnostic apparatus is capable of reducing the influence of noise on each ultrasonic image and definitely displaying the biopsy needle in the ultrasonic image.

The invention of a fifth aspect provides an ultrasonic diagnostic apparatus according to the above invention wherein the superimposed-image generating means extracts the ultrasonic images highest in luminance on every pixel from the ultrasonic images stored in the ultrasonic image storing means.

According to the invention of the fifth aspect, since ones highest in luminance are extracted from the ultrasonic images every pixels and superimposed on one another, the ultrasonic diagnostic apparatus is capable of definitely displaying the biopsy needle in the ultrasonic image.

The invention of a sixth aspect provides an ultrasonic diagnostic apparatus according to the above invention, further comprising setting changing means which accepts a predetermined change in setting from a user, wherein the superimposed-image generating means superimposes ultrasonic images on one another on the basis of the settings changed by the setting changing means.

According to the invention of a sixth aspect, since the change in setting from the user is accepted, the ultrasonic diagnostic apparatus is capable of accepting, for example, such a setting as to add ultrasonic images in time units or frame units or such a setting as to automatically perform image processing after a condition input has been received from the user in advance.

The invention of a seventh aspect provides an ultrasonic diagnostic apparatus which applies ultrasound to within a body to be examined through an ultrasound probe, receives ultrasonic sounds reflected from within the body and a biopsy needle inserted in the body by means of the ultrasound probe and sequentially generates ultrasonic images, comprising: ultrasonic radiation control means which applies the ultrasound vertically to the biopsy needle being inserted in the body; image converting means which converts a reflected signal of the biopsy needle obtained by the ultrasonic radiation control means to an image; and image generating means which generates an image corresponding to the biopsy needle imaged by the image converting means.

According to the invention of the seventh aspect, since the ultrasound is applied vertically to the biopsy needle inserted in the body, the ultrasonic diagnostic apparatus is capable of obtaining a strong reflected signal from the biopsy needle and definitely displaying the biopsy needle in the corresponding ultrasonic image.

The invention of an eighth aspect provides an ultrasonic diagnostic apparatus according to the above invention wherein the ultrasonic radiation control means applies the ultrasound vertically to a target line displayed on the corresponding ultrasonic image by mounting an attachment to the ultrasound probe.

According to the invention of the eighth aspect, since the ultrasound is applied vertically to the target line corresponding to the guide for inserting the biopsy needle in the body, the ultrasonic diagnostic apparatus is capable of applying the ultrasound substantially perpendicular to the biopsy needle.

The invention of a ninth aspect provides an ultrasonic diagnostic apparatus according to the above invention, further comprising position detecting means which specifies a position of the biopsy needle, wherein the ultrasonic radiation control means applies the ultrasound vertically to the position specified by the position detecting means.

According to the invention of the ninth aspect, since the position is specified by the detecting means (sensor, for example) attached to the biopsy needle, the ultrasonic diagnostic apparatus is capable of applying an ultrasonic signal substantially vertically to the biopsy needle.

The invention of a tenth aspect provides an ultrasonic diagnostic apparatus according to the above invention wherein the ultrasonic radiation control means applies the ultrasound in plural directions while the biopsy needle is being inserted in the body and further applies the ultrasound in the direction in which a strong reflected signal is obtained.

According to the invention of the tenth aspect, since the ultrasound is applied in the plural directions in advance and applied in the direction in which the strong reflected signal is obtained, the ultrasonic diagnostic apparatus is capable of applying the ultrasound substantially perpendicular to the biopsy needle without using a biopsy guide and a sensor.

The invention of an eleventh aspect provides an ultrasonic diagnostic apparatus according to the above invention, further comprising setting changing means which accepts a predetermined change in setting from a user, wherein the ultrasonic radiation control means applies the ultrasound on the basis of the settings changed by the setting changing means.

According to the invention of the eleventh aspect, since the ultrasonic diagnostic apparatus accepts the user's change in setting about the radiation direction of an ultrasonic signal, the user is able to freely change the direction of ultrasonic radiation where the position of the biopsy needle can be confirmed (confirmed by a position sensor or visually, for example), and thereby to make an adjustment such that the ultrasound is applied vertically to the biopsy needle.

The invention of a twelfth aspect provides an ultrasonic diagnostic apparatus according to the above invention wherein the ultrasonic radiation control means further includes combination generating means which applies the ultrasound vertically to the biopsy needle to generate image data of the biopsy needle and applies the ultrasound to the body to generate image data in the body, and which combines the image data in the body and the image data of the biopsy needle to generate an image.

According to the invention of the twelfth aspect, since the in-vivo image and the image in the neighborhood of the biopsy needle are displayed in combination, the ultrasonic diagnostic apparatus is capable of safely performing paracentesis or puncture.

The invention of a thirteenth aspect provides an ultrasonic diagnostic apparatus according to the above invention wherein each of the ultrasonic images is a two-dimensional or three-dimensional ultrasonic image.

According to the invention of the thirteenth aspect, the ultrasonic diagnostic apparatus is capable of safely effecting paracentesis even on the three-dimensional ultrasonic image as well as the two-dimensional ultrasonic image.

The invention of a fourteenth aspect provides an ultrasonic image generating method for applying ultrasound to within a body to be examined through an ultrasound probe, receiving ultrasonic sounds reflected from within the body and a biopsy needle inserted in the body by means of the ultrasound probe and sequentially generating ultrasonic images, comprising the steps of sequentially storing the ultrasonic images, sequentially superimposing the stored ultrasonic images in order of generation thereof, and generating the superimposed image.

According to the invention of the fourteenth aspect, since the ultrasonic images are superimposed on one another to generate the image and the generated image is displayed, the ultrasonic image generating method is capable of definitely displaying the biopsy needle inserted in the body (in which the biopsy needle is displayed by snatches, for example) in which it is hard to be well displayed, in the ultrasonic image.

The invention of a fifteenth aspect provides an ultrasonic image generating program for allowing a computer to execute a method for applying ultrasound to within a body to be examined through an ultrasound probe, receiving ultrasonic sounds reflected from within the body and a biopsy needle inserted in the body by means of the ultrasound probe and sequentially generating ultrasonic images, comprising an ultrasonic image storing procedure for sequentially storing the ultrasonic images, an ultrasonic image superimposing procedure for sequentially superimposing the ultrasonic images stored in ultrasonic image storing means in order of generation thereof, and a superimposed-image generating procedure for generating an image superimposed by ultrasonic image superimposing means, wherein the computer is allowed to execute the ultrasonic image storing procedure, the ultrasonic image superimposing procedure and the superimposed-image generating procedure.

According to the invention of the fifteenth aspect, since the ultrasonic images are superimposed on one another to generate the image and the generated image is displayed, the ultrasonic image generating program is capable of definitely displaying the biopsy needle inserted in the body (in which the biopsy needle is displayed by snatches, for example) in which it is hard to be well displayed, in the ultrasonic image.

The invention of a sixteenth aspect provides an ultrasonic image generating method for applying ultrasound to within a body to be examined through an ultrasound probe, receiving ultrasonic sounds reflected from within the body and a biopsy needle inserted in the body by means of the ultrasound probe and sequentially generating ultrasonic images, comprising the steps of applying the ultrasound vertically to the biopsy needle inserted in the body, converting a reflected signal of the biopsy needle obtained by ultrasonic radiation control means to an image, and generating an image corresponding to the biopsy needle imaged by image converting means.

According to the invention of the sixteenth aspect, since the ultrasound is applied vertically to the biopsy needle inserted in the body, the ultrasonic image generating method is capable of obtaining a strong reflected signal from the biopsy needle and definitely displaying the biopsy needle in the ultrasonic image.

The invention of a seventeenth aspect provides an ultrasonic image generating program for allowing a computer to execute a method for applying ultrasound to within a body to be examined through an ultrasound probe, receiving ultrasonic sounds reflected from within the body and a biopsy needle inserted in the body by means of the ultrasound probe and sequentially generating ultrasonic images, comprising an ultrasonic radiation control procedure for applying the ultrasound vertically to the biopsy needle inserted in the body, an image converting procedure for converting a reflected signal of the biopsy needle obtained by ultrasonic radiation control means to an image, and an image generating procedure for generating an image corresponding to the biopsy needle imaged by image converting means, wherein the computer is allowed to execute the ultrasonic radiation control procedure, the image converting procedure and the image generating procedure.

According to the invention of the seventeenth aspect, since the ultrasound is applied vertically to the biopsy needle inserted in the body, the ultrasonic image generating program is capable of obtaining a strong reflected signal from the biopsy needle and definitely displaying the biopsy needle in the corresponding ultrasonic image.

According to the present invention, each of the ultrasonic diagnostic apparatus, the ultrasonic image generating method and the ultrasonic image generating program is capable of definitely displaying a biopsy needle inserted in a body (in which the biopsy needle is displayed by snatches, for example) in which it is hard to be well displayed, in its corresponding ultrasonic image since ultrasonic images are superimposed on one another to generate an image and the generated image is displayed.

According to the present invention as well, each of the ultrasonic diagnostic apparatus, the ultrasonic image generating method and the ultrasonic image generating program is capable of obtaining a strong reflected signal from a biopsy needle and definitely displaying the biopsy needle in the corresponding ultrasonic image since ultrasound is applied vertically to the biopsy needle inserted in a body.

The ultrasonic diagnostic apparatus, the ultrasonic image generating method and the ultrasonic image generating program according to the present invention are effective for the case in which ultrasound is applied to within a sample or body to be examined through an ultrasound probe, ultrasonic sounds reflected from within the body and a biopsy needle inserted in the body are received at the ultrasound probe, and ultrasonic images are sequentially generated. They are particularly suitable for definite display of the biopsy needle in each ultrasonic image.

Further objects and advantages of the present invention will be apparent from the following description of the preferred embodiments of the invention as illustrated in the accompanying drawings.

### BRIEF DESCRIPTION OF THE DRAWINGS

FIG. 1 is a diagram for describing a general outline of an ultrasonic diagnostic apparatus according to a first embodiment.

FIG. 2 is a block diagram showing the configuration of the ultrasonic diagnostic apparatus according to the first embodiment.

FIG. 3 is a diagram illustrating images stored in an image storage unit according to the first embodiment.

FIG. 4 is a flowchart showing the flow of a biopsy needle image generating process according to the first embodiment.

FIG. 5 is a diagram for describing a general outline of an ultrasonic diagnostic apparatus according to a second embodiment.

FIG. 6 is a block diagram showing the configuration of the ultrasonic diagnostic apparatus according to the second embodiment.

FIG. 7 is a flowchart illustrating the flow of an image storing process according to the second embodiment.

FIG. 8 is a flowchart showing the flow of an image generating process according to the second embodiment.

FIG. 9 is a diagram illustrating a computer which executes an ultrasonic image generating program according to the first embodiment.

FIG. 10 is a diagram depicting a computer which executes an ultrasonic image generating program according to the second embodiment.

### DETAILED DESCRIPTION OF THE INVENTION

Embodiments of ultrasonic diagnostic apparatuses, ultrasonic image generating methods and ultrasonic image generating programs according to the present invention will hereinafter be explained in detail with reference to the accompanying drawings. Incidentally, the ultrasonic diagnostic apparatus will be explained below as a first embodiment, and other embodiments contained in the present invention will hereinafter be described as a second embodiment and a third embodiment.

### First Embodiment

In the first embodiment shown below, the outline and features of the ultrasonic diagnostic apparatus according to the first embodiment, and the configuration of the ultrasonic diagnostic apparatus and the flow of processing thereof will be explained in order, and advantages obtained by the first embodiment will finally be explained.

Outline and Features (First Embodiment)

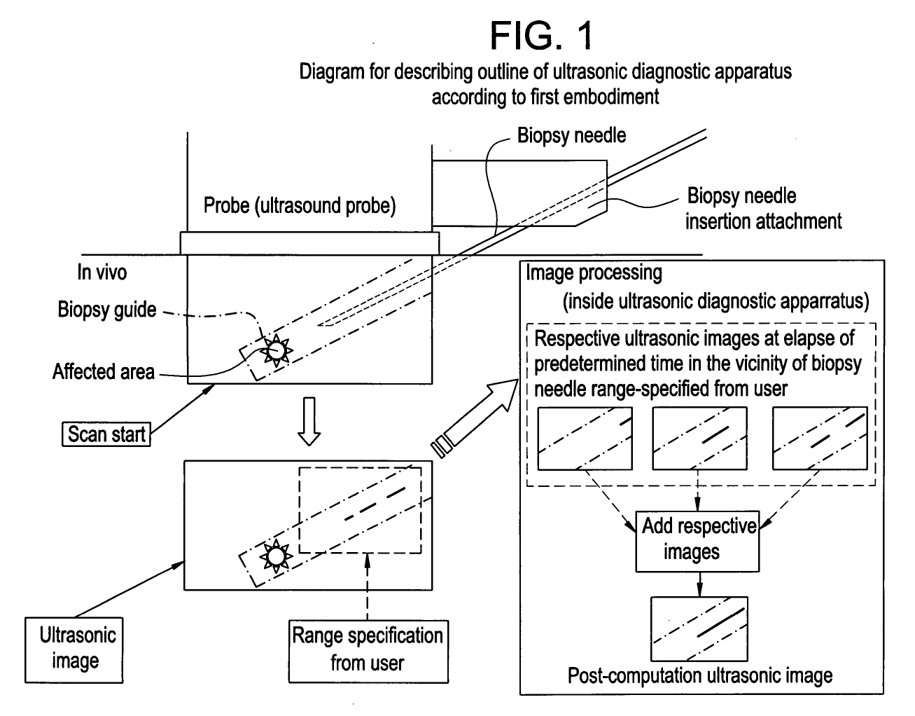

To begin with, the general outline and features of the ultrasonic diagnostic apparatus according to the first embodiment will be explained using FIG. 1. FIG. 1 is a diagram for describing the outline of the ultrasonic diagnostic apparatus according to the first embodiment. As shown in the same figure, the ultrasonic diagnostic apparatus brings a probe connected thereto into contact with a body surface to apply ultrasound, receives signals reflected from in vivo and a biopsy needle inserted into a body and generates ultrasonic images (tomograms) in the body and at the biopsy needle on the basis of the received signals.

A major feature of the ultrasonic diagnostic apparatus having such a general outline is that it generates the ultrasonic images in superimposed form and displays the generated image thereon. It is thus possible to definitely display the biopsy needle on the displayed ultrasonic image.

The major feature referred to above will be explained in brief. When the ultrasonic diagnostic apparatus starts scan in the directly-below direction orthogonal to the body surface while inserting the biopsy needle into the body, an image at a specific section in the body, an image of an affected area corresponding to a target for paracentesis, and a biopsy guide are represented on ultrasonic images.

Here, the biopsy guide displays, on the ultrasonic image, an expected path of the biopsy needle upon executing biopsying by using an attachment for biopsy needle insertion in the probe. A user directs the biopsy guide to a patent and fine-adjusts it while the probe is being moved, and inserts the biopsy needle into the body along the biopsy guide. And the ultrasonic diagnostic apparatus receives signals reflected from in vivo and the biopsy needle inserted in the body and sequentially stores therein generated ultrasonic images simultaneously with the start of scanning.

Subsequently, when the ultrasonic diagnostic apparatus accepts range specification (e.g., the designation of a cursor or the like on a display) in the vicinity (where, for example, images of the biopsy needle, which are generated by snatches, and kinking of in vivo tissue on the periphery of the inserted biopsy needle are confirmed) of insertion of the biopsy needle on the ultrasonic image displayed on an output unit (e.g., display), the ultrasonic diagnostic apparatus reads out the corresponding ultrasonic image stored immediately before the reception of the range specification from the sequentially stored ultrasonic images (reads them with time such as a few second ago being taken as an index or with the number of frames as being designated).

Next, picture elements are added every pixels with respect to range specified portions of these ultrasonic images to thereby create an ultrasonic image obtained by superimposing the respective pixels. Even while the superimposed ultrasonic image is being created, ultrasonic images are sequentially stored. The superimposed ultrasonic image and the latest ultrasonic image are generated in combination and displayed on the output unit.

From this point of view, the ultrasonic diagnostic apparatus sequentially lap-generates the ultrasonic images of the biopsy needle inserted in the body (in which they are generated by snatches through the biopsy needle, for example) in which they are hard to be well generated, with respect to the biopsy-needle peripheral range in which the designation given from the user has been accepted. Hence the ultrasonic diagnostic apparatus is capable of definitely displaying the biopsy needle in the ultrasonic image.

[Configuration of Ultrasonic Diagnostic Apparatus (First Embodiment)]

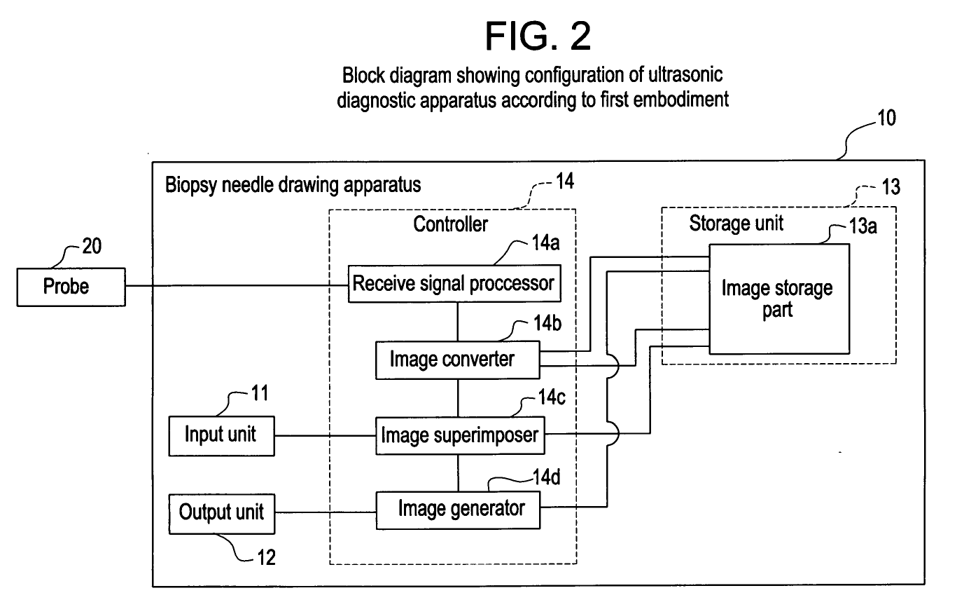

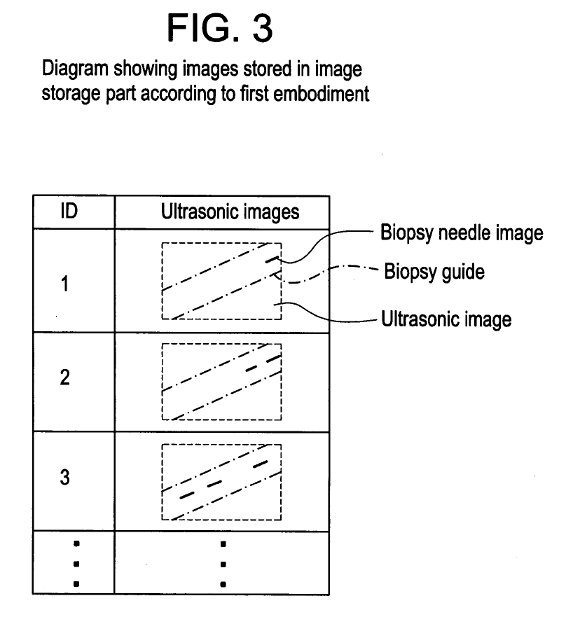

The configuration of the ultrasonic diagnostic apparatus 10 according to the first embodiment will next be explained using FIGS. 2 and 3. FIG. 2 is a block diagram showing the configuration of the ultrasonic diagnostic apparatus 10 according to the first embodiment. FIG. 3 is a diagram showing images stored in an image storage unit according to the first embodiment. As shown in FIG. 2, the ultrasonic diagnostic apparatus 10 comprises an input unit 11, an output unit 12, a storage unit 13 and a controller 14.

Of these, the input unit 11 is an input means for receiving inputs such as various information therein. Specifically, the input unit 11 accepts and inputs an image processing range or the like at an image superimposer 14c to be described later. The output unit 12 is an output means for outputting various information and is configured with being equipped with a monitor (or a display or a touch panel). Specifically, the output unit 12 displays and outputs an ultrasonic image at a specific section in a body, a biopsy guide, etc.

The storage unit 13 is a storing means (memory means) for storing data and programs necessary for various processes by the controller 14. As one closely related to the present invention in particular, the storage unit 13 is provided with an image storage part 13a.

The ultrasonic image storage part 13a is a means for storing ultrasonic images obtained by converting signals reflected from in vivo and a biopsy needle inserted in the body by an image converter 14b to be described later. Specifically, as illustrated in FIG. 3, the ultrasonic images generated by receiving the reflected signals at the image converter 14b are sequentially stored with being marked with IDs simultaneously with the start of scanning.

The controller 14 is a processor which has an internal memory for storing control programs such as an OS, etc., programs which define various processing procedures or the like, and required data and which executes various processes by them. As ones closely related to the present invention in particular, the controller 14 is provided with a receive signal processor 14a, the image converter 14b, an image superimposer 14c, and an image output part 14d. Incidentally, the image superimposer 14c corresponds to “ultrasonic image superimposing means” as defined in claims, and the image generator 14d similarly corresponds to “ultrasonic image generating means”.

Of the controller 14, the receive signal processor 14a is a processor which receives reflected signals sent from a probe 20 and relays the same to the image converter 14b. Described specifically, the receive signal processor 14a accepts ultrasonic signals reflected from in vivo and the biopsy needle inserted in the body and effects signal processing such as amplification, detection, etc. thereon, and outputs the same to the image converter 14b.

The image converter 14b is a processor which image-converts each signal sent from the receive signal processor 14a. Specifically, the image converter 14b converts the signal sent from the receive signal processor 14a to an ultrasonic tomographic image and outputs the same to the image generator 14d and the storage unit 13.

The image superimposer 14c is a processor which effects image processing on the ultrasonic image generated at the output unit 12. Specifically, when the input unit 11 accepts the designation of an image processing range from a user, the image superimposer 14c reads out the ultrasonic images stored in the image storage part 13a (reads them from the images stored inside the apparatus, which are illustrated in FIG. 3 by way of example, with time such as a few second ago being taken as an index or with the number of frames as being designated), adds picture elements every pixels with respect to user-designated ranges at the respective ultrasonic images, and outputs an ultrasonic image obtained by superimposing the respective pixels to the image generator 14d.

The image generator 14d is a processor which generates the ultrasonic image at the output unit 12. Specifically, the image generator 14d combines the ultrasonic tomographic image outputted from the image converter 14b and the latest ultrasonic tomographic image read from the image storage part 14a and generates the combined one for the output unit 12.

[Biopsy Needle Image Generating Processing (First Embodiment)]

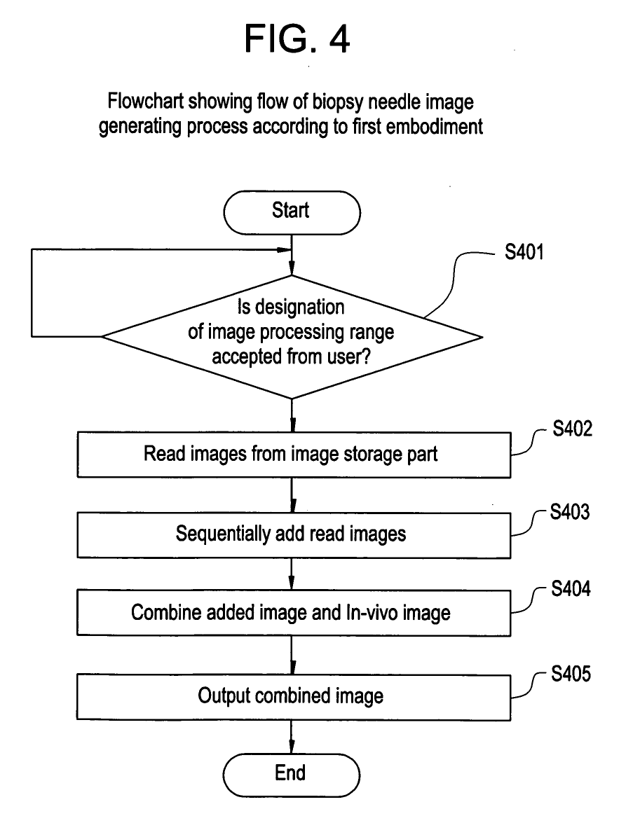

A description will next be made of a biopsy needle image generating process according to the first embodiment using FIG. 4. FIG. 4 is a flowchart showing the flow of the biopsy needle image generating process according to the first embodiment.

When the designation of an image processing range from a user is accepted via the input unit 11 as shown in the same figure (when the answer is Yes in Step S401), the image superimposer 14c reads out the ultrasonic images stored immediately before the reception of the designation from the user from the image storage part 13a (Step S402). Next, the image superimposer 14c adds picture elements every pixels with respect to the user-designated ranges at the respective ultrasonic images to thereby generate an ultrasonic image in which the respective pixels are superimposed on one another (Step S403), and outputs the generated ultrasonic image to the image generator 14d.

Subsequently, the image generator 14d reads the latest ultrasonic image from the image storage part 13a in response to the output of the ultrasonic image generated at the image superimposer 14c and combines the read ultrasonic image and the ultrasonic image generated at the image superimposer 14c (Step S404). Then, the combined image generated from the image generator 14d is outputted to the output unit 12 (Step S405). The ultrasonic diagnostic apparatus 10 terminates the biopsy needle image generating process. Incidentally, the processing described above is repeatedly performed as long as the designation of each image processing range from the user is received.

[Advantages of First Embodiment]

According to the first embodiment as described above, since the ultrasonic image is generated by superimposition, the ultrasonic diagnostic apparatus 10 is capable of sequentially superimposing the ultrasonic images of the biopsy needle inserted in the body in which they are hard to be well generated (in which the images of the biopsy needle are generated by snatches, for example) and thereby definitely displaying the biopsy needle in the resultant ultrasonic image.

According to the first embodiment as well, since the images around the biopsy needle are lap-generated, the ultrasonic diagnostic apparatus 10 is capable of definitely displaying only the periphery of the biopsy needle in the ultrasonic image.

Further, according to the first embodiment, since the ultrasonic images are superimposed by addition every pixels, the ultrasonic diagnostic apparatus 10 is capable of definitely displaying the biopsy needle in the ultrasonic image in accordance with a relatively simple process.

### Second Embodiment

Incidentally, although the first embodiment has explained the ultrasonic diagnostic apparatus which effects the image processing on the ultrasonic images near the biopsy needle, the present invention is not limited to it. The direction of radiation of the ultrasonic signal may be controlled so as to display the biopsy needle on the ultrasonic image.

Thus, in the second embodiment shown below, the outline and features of an ultrasonic diagnostic apparatus according to the second embodiment, and the configuration of the ultrasonic diagnostic apparatus and the flow of processing thereof will be explained in order, and advantages obtained by the second embodiment will finally be explained.

[Outline and Features]

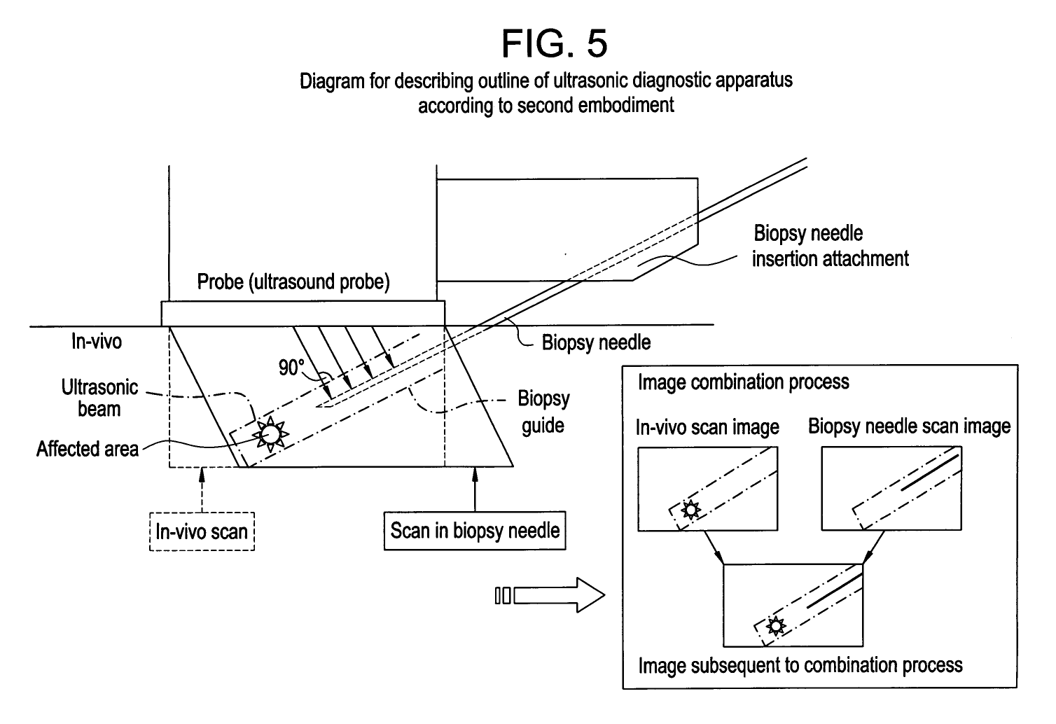

To begin with, the general outline and features of the ultrasonic diagnostic apparatus according to the second embodiment will be explained using FIG. 5. FIG. 5 is a diagram for describing the outline of the ultrasonic diagnostic apparatus according to the second embodiment. As shown in the same figure, the ultrasonic diagnostic apparatus brings a probe connected thereto into contact with a body surface to apply an ultrasound signal in a predetermined cycle, receives signals reflected from in vivo and a biopsy needle inserted into a body and generates ultrasonic images (tomograms) in the body and at the biopsy needle on the basis of the received signals.

A major feature of the ultrasonic diagnostic apparatus having such a general outline is that the ultrasonic signal is applied vertically to the biopsy needle inserted in the body. Thus, the ultrasonic diagnostic apparatus is capable of accepting a strong reflected signal from the biopsy needle and generating and displaying the ultrasonic image of the biopsy needle.

The major feature referred to above will be explained in brief. When the ultrasonic diagnostic apparatus starts in-vivo scan for applying an ultrasonic signal from the probe while the biopsy needle is being inserted into the body, an image at a specific section in the body, an image of an affected area corresponding to a target for paracentesis, and a biopsy guide are represented on ultrasonic images. Here, the biopsy guide is used to display, on the ultrasonic image, an expected path of the biopsy needle upon executing biopsying by using an attachment for biopsy needle insertion in the probe. A user directs the biopsy guide to a patent and fine-adjusts it while the probe is being moved, and inserts the biopsy needle into the body along the biopsy guide. And the ultrasonic diagnostic apparatus temporarily stores in-vivo images obtained by the in-vivo scan.

Although the biopsy needle inserted in the body is not well displayed as viewed in the direction of irradiation of ultrasound at the in-vivo scan, the user changes the radiation direction of the ultrasonic signal to the direction orthogonal to the biopsy guide at the position where the biopsy needle is inserted in the body to some degree while the rough position of the biopsy needle is being confirmed due to kinking of a in vivo tissue or the like, for example. In doing so, the ultrasonic signal is applied substantially vertically to the biopsy needle from the probe since the biopsy needle is inserted into the body along the biopsy guide. As a result, a strong reflected signal is accepted from the biopsy needle through the probe and converted to an image, and the resultant image of the biopsy needle is temporarily stored. Subsequently, the ultrasonic diagnostic apparatus reads the previously stored in-vivo image and the biopsy needle image and combines these images together to thereby generate the combined one, and displays it on the output unit (e.g., display).

Incidentally, the irradiation of the ultrasound is alternately carried out in the normal direction and in the direction of the biopsy guide. Then, images obtained from the respective directions may finally be displayed on the output unit in combination. For example, one or plural sound ray radiation for obtaining a normal image, and one or plural sound ray radiation for obtaining a biopsy needle image may alternately be carried out. The radiation of sound rays may not be switched alternately every once. The sound-ray radiation for obtaining the normal image and the sound-ray radiation for obtaining the biopsy needle image may alternately be performed by switching every plural times.

In view of this, the ultrasonic diagnostic apparatus is capable of obtaining a strong reflected signal from the biopsy needle since the ultrasound is applied vertically to the biopsy needle inserted in the body, and clearly displaying the biopsy needle in the ultrasonic image.

[Configuration of Ultrasonic Diagnostic Apparatus (Second Embodiment)]

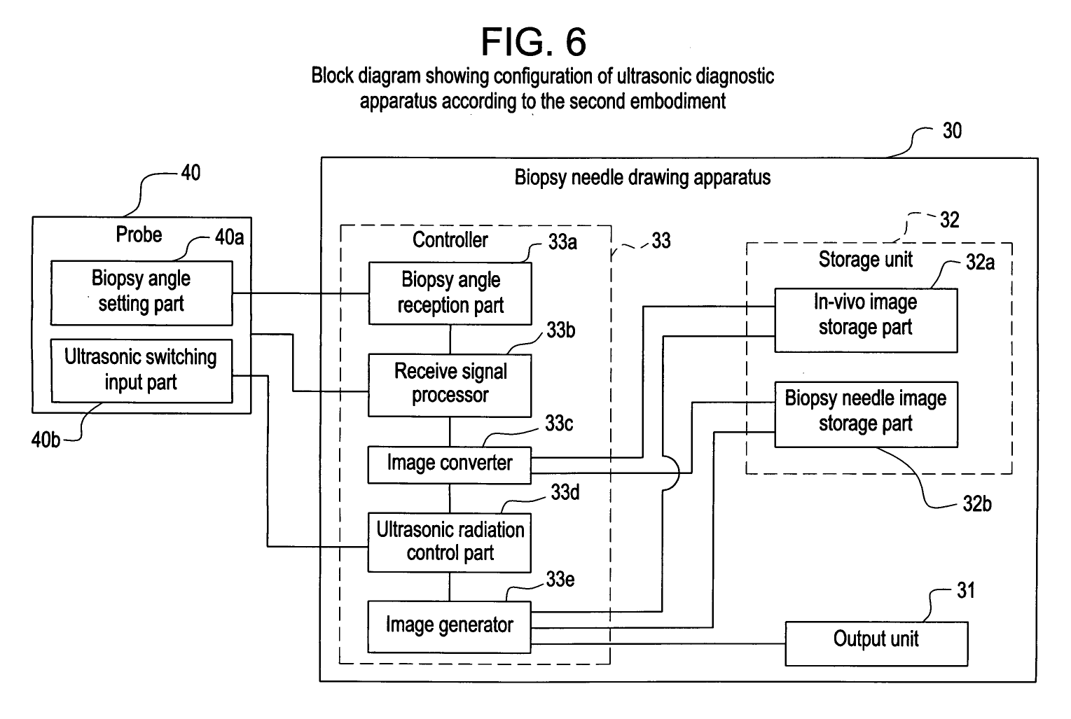

A configuration of an ultrasonic diagnostic apparatus according to a second embodiment will next be explained using FIG. 6. FIG. 6 is a block diagram showing the configuration of the ultrasonic diagnostic apparatus according to the second embodiment. As shown in the same figure, the ultrasonic diagnostic apparatus 30 comprises an output unit 31, a storage unit 32 and a controller 33.

Of these, the output unit 31 is an output means for outputting various information and is configured with being equipped with a monitor (or a display or a touch panel). Specifically, the output unit 31 displays and outputs an ultrasonic image at a specific section in the body, a biopsy guide, etc.

The storage unit 32 is a storing means (memory means) for storing data and programs necessary for various processes by a controller 33. As one closely related to the present invention in particular, the storage unit 32 is provided with an in-vivo image storage part 32a and a biopsy needle image storage part 32b.

Of the storage unit 32, the in-vivo image storage part 32a is a storing means for storing an ultrasonic image obtained by in-vivo scan. Specifically, the in-vivo image storage part 32a stores therein an ultrasonic tomographic image obtained by applying ultrasound in the normal operation. The biopsy needle image storage part 32b is a means for storing an ultrasonic image obtained by scan relative to the biopsy needle. Specifically, the biopsy needle image storage unit 32b stores therein a biopsy needle image obtained by applying ultrasound to the biopsy guide displayed on the ultrasonic image.

The controller 33 is a processor which has an internal memory for storing control programs such as an OS, etc., programs which define various processing procedures or the like, and required data and which executes various processes by them. As ones closely related to the present invention in particular, the controller 33 is provided with a biopsy angle reception part 33a, a receive signal processor 33b, an image converter 33c, an ultrasonic radiation control part 33d and an image generator 33e. Incidentally, the ultrasonic radiation control part 33d corresponds to “ultrasonic radiation control means” as defined in claims, the image converter 33c similarly corresponds to “image converting means”, and the image generator 33e similarly corresponds to “image generating means”.

Of the controller 33, the biopsy angle reception part 33a is a means for accepting the angle of the biopsy guide displayed on the ultrasonic image. Specifically, the biopsy angle reception part 33a receives a signal automatically sent from a biopsy angle setting part 40a with an attachment (attachment for biopsy needle insertion, for example) being mounted to a probe 40.

The receive signal reception part 33b is a processor for accepting various signals sent from the probe 40 and relaying them to the image converter 33c. Specifically, the receive signal reception part 33b relays each signal sent from the biopsy angle reception part 40a to the image converter 33c. Also the receive signal reception part 33b receives reflected signals sent from in vivo and the biopsy needle inserted in the body and relays the same to the image converter 33c.

The image converter 33c is a processor for image-converting each signal sent from the receive signal processor 33b. Specifically, the image converter 33c converts the corresponding signal sent from the biopsy angle reception part 33a via the receive signal processor 33b to a biopsy guide image and outputs it to the image generator 33e. The image converter 33c converts the reflected signals from in vivo and the biopsy needle inserted in the body, which are sent via the receive signal processor 33b, to ultrasonic images and outputs the same to the in-vivo image storage part 32a and the biopsy needle image storage part 32b.

The ultrasonic radiation control part 33d is a processor for controlling the direction of radiation of ultrasound applied from the probe 40. Specifically, when the ultrasonic radiation control part 33d receives an input for switching the ultrasonic radiation direction from a user via an ultrasonic switching input part 40b of the probe 40, the ultrasonic radiation control part 33d switches the radiation direction of the ultrasound from the direction orthogonal to a body surface to the direction vertical to the biopsy guide and applies the ultrasound.

The image generator 33e is a processor for generating an ultrasonic image for the output unit 31. Specifically, the image generator 33e combines the images read from the in-vivo image storage part 32a and the biopsy needle image storage part 32b and generates the combined one for the output unit 31.

[Image Storing Process (Second Embodiment)]

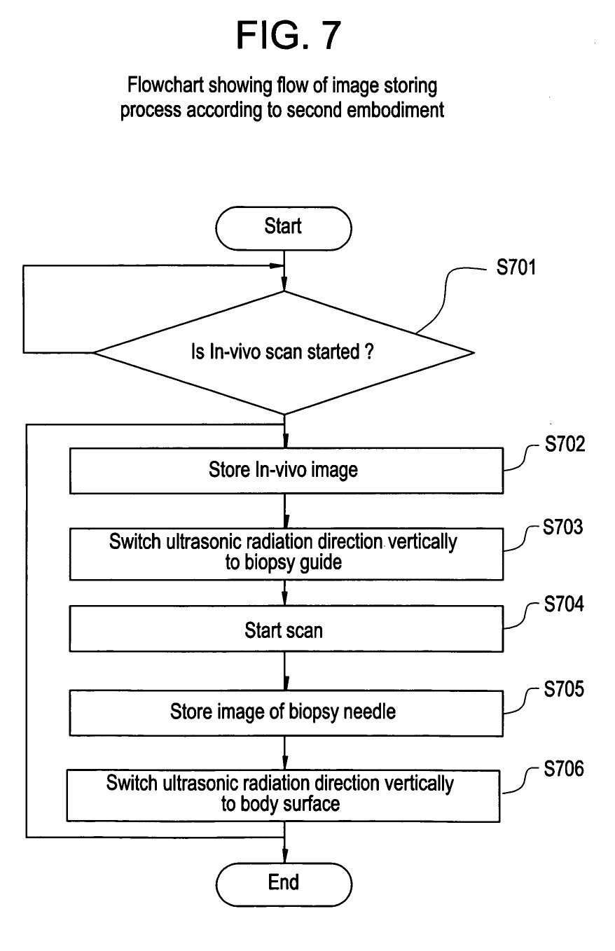

The flow of an image storing process according to the second embodiment will next be explained using FIG. 7. FIG. 7 is a flow chart showing the flow of the image storing process according to the second embodiment.

When the ultrasonic diagnostic apparatus 30 is activated to start in vivo scan while the biopsy needle is being inserted with the biopsy guide displayed on the output unit 31 as a standard in a state in which the probe 40 is being brought into contact with the body surface (when the answer is Yes in Step S701), the reflected signal sent from in vivo is converted into an ultrasonic tomographic image, which is temporarily stored in the in-vivo image storage part 32a (Step S702).

Subsequently, when the ultrasonic radiation control part 33d accepts an ultrasonic radiation direction switching input from the user via the ultrasonic switching input part 40b, the ultrasonic radiation control part 33d switches the direction of radiation of the ultrasound from the direction orthogonal to the body surface to the direction vertical to the biopsy guide and applies the ultrasound (Step S703). With the switching of the ultrasonic radiation direction, the ultrasonic radiation control part 33d starts scan in the direction orthogonal to the biopsy guide (Step S704).

In doing so, the ultrasound is applied vertically to the biopsy needle or substantially vertically thereto because the user inserts the biopsy needle into the body along the biopsy guide. As a result, a strong reflected signal is received from the biopsy needle via the probe and then converted to an image, after which a resultant biopsy needle image is temporarily stored in the biopsy needle image storage part 32b (Step S705).

When the user switches the radiation direction of the ultrasound to the direction orthogonal to the body surface again (Step S706), the ultrasonic diagnostic apparatus 30 temporarily terminates the image storing process. Incidentally, the processing described as above is repeatedly performed so long as the user continues scanning.

[Image Generating Process (Second Embodiment)]

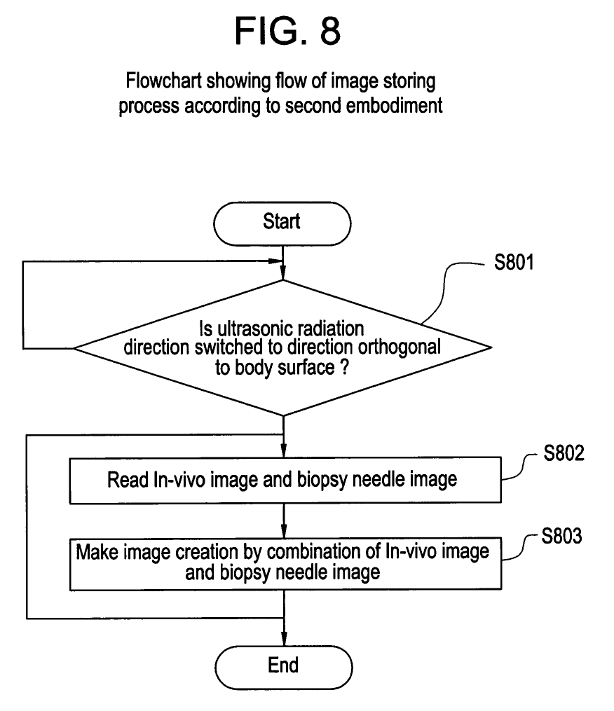

The flow of an image generating process according to the second embodiment will subsequently be explained using FIG. 8. FIG. 8 is a flowchart showing the flow of the image generating process according to the second embodiment.

When the input for switching of the ultrasonic radiation direction (corresponding to the direction orthogonal to the body surface) is accepted from the user via the ultrasonic switching input part 40b of the probe 40 as shown in the same figure (Step S801), the image generator 33e reads the corresponding in-vivo ultrasonic tomographic image from the in-vivo image storage part 32a and reads the corresponding biopsy needle image from the biopsy needle image storage part 33b (Step S802). And the image generator 33e combines these images together to generate an image and outputs it to the output unit 31 (Step S803). Hence, the ultrasonic diagnostic apparatus terminates the image generating process. Incidentally, the processing described above is repeatedly performed so long as the user continues to scan.

[Advantages of Second Embodiment]

According to the second embodiment as described above, since the ultrasound is applied vertically to the biopsy needle inserted into the body, the ultrasonic diagnostic apparatus 30 is capable of obtaining a strong reflected signal from the biopsy needle and definitely displaying the biopsy needle in the ultrasonic image.

According to the second embodiment as well, since the ultrasound is applied vertically to a target line corresponding to the guide for inserting the biopsy needle into the body, the ultrasonic diagnostic apparatus 30 is capable of applying the ultrasound substantially perpendicular to the biopsy needle.

Further, according to the second embodiment, since the in-vivo image and the image in the vicinity of the biopsy needle are combined together to generate the image, the ultrasonic diagnostic apparatus 30 is capable of safely performing paracentesis.

### Third Embodiment

Now, although the ultrasonic diagnostic apparatuses according to the first and second embodiments have been explained so far, the present invention may be effected on various different forms even except for the first and second embodiments. Thus, various different forms will respectively be explained below in sorted forms of (1) through (9) as a third embodiment.

(1) Average of Ultrasound Image

Although the first embodiment has explained the case in which the ultrasonic images are added every pixels and superimposed on one another, the present invention is not limited to it. The ultrasonic images may be superimposed on the average every pixels. Thus, the ultrasonic diagnostic apparatus is capable of reducing the influence of noise on each ultrasonic image and definitely displaying the biopsy needle in the ultrasonic image.

(2) Maximum Value Luminance Projection of Ultrasound Image

Although the first embodiment has explained the case in which the ultrasonic images are added every pixels and superimposed on one another, the present invention is not limited to it. Ones highest in luminance are extracted from the ultrasonic images every pixels and may be superimposed on one another. Thus, the ultrasonic diagnostic apparatus is capable of definitely displaying the biopsy needle in the ultrasonic image.

(3) Reception of Changes in Setting in Image Processing

Although the first embodiment has explained the case in which the range specification is accepted from the user and the image processing is performed, the present invention is not limited to it. It may be feasible to accept changes in setting from the user and carry out the image processing on the basis of such changed settings. It is possible to accept, for example, such a setting as to add ultrasonic images in time units or frame units or such a setting as to automatically perform image processing after a condition input has been received from the user in advance.

(4) Specify Position of Biopsy Needle and Apply Ultrasound

Although the second embodiment has explained the case in which the ultrasound is applied vertically to the biopsy guide, the present invention is not limited to it. It may be feasible to mount a position detecting means (e.g., position sensor) to the biopsy needle to specify its position and apply ultrasound. Thus, the ultrasonic diagnostic apparatus is capable of applying the ultrasound approximately perpendicular to the biopsy needle.

(5) Apply Ultrasound in Plural Directions in Advance

Although the second embodiment has described the case in which the ultrasound is applied perpendicular to the biopsy guide, the present invention is not limited to it. It may be feasible to apply the ultrasound in plural directions in advance and apply the ultrasound in the direction in which a strong reflected signal has been obtained. It is thus possible to apply the ultrasound substantially perpendicular to the biopsy needle without using the biopsy guide and the sensor.

(6) Reception of Changes in Setting of Ultrasonic Radiation

Although the second embodiment has explained the case in which when the input for switching of the ultrasonic radiation direction is accepted from the user, the ultrasound is applied vertically to the biopsy guide, the present invention is not limited to it. It may be feasible to cause the user to change the setting of the radiation direction of the ultrasound. Thus, when the position of the biopsy needle can be confirmed (it is confirmed by a position senor or visually, for example), the user is able to freely change the direction of ultrasonic radiation and perform fine adjustments such that the ultrasound is applied vertically to the biopsy needle.

(7) Dimension of Ultrasound Image

Although the above embodiments have explained the case in which each ultrasonic image is two-dimensional, the present invention is not limited to it. The ultrasonic images may be three-dimensional. Thus, the ultrasonic diagnostic apparatus is capable of safely carrying out paracentesis even in the three-dimensional ultrasonic images.

(8) Apparatus Configuration

The respective constituent elements of the ultrasonic diagnostic apparatus 10 shown in FIG. 2 are conceptual in function. They do not necessarily require such configurations as illustrated in the drawing physically. That is, the specific forms of dispersion and integration of the ultrasonic diagnostic apparatus 10 are not limited to such ones as shown in the figure. All or some of the respective constituent elements can be configured so as to be functionally or physically dispersed/integrated in arbitrary units, depending upon various loads and use conditions or the like, as in the case in which, for example, the image converter 14b, the image superimposer 14c and the image generator 14d are integrated into one. Further, all or arbitrary some of the respective processing functions carried out by the respective devices can be implemented by a CPU or programs analyzed and executed by the CPU, or realized as hardware by wired logic.

Similarly even to the ultrasonic diagnostic apparatus 30 shown in FIG. 6, all or some of the respective constituent elements can be configured so as to be functionally or physically dispersed/integrated in arbitrary units, depending upon various loads and use conditions or the like, as in the case in which, for example, the biopsy angle reception part 33b and the receive signal processor 33b are integrated into one, or the image converter 33c and the image generator 33e are integrated into one. Further, all or arbitrary some of the respective processing functions carried out by the respective devices can be implemented by a CPU or programs analyzed and executed by the CPU, or realized as hardware by wired logic.

(9) Biopsy Needle Generating Program

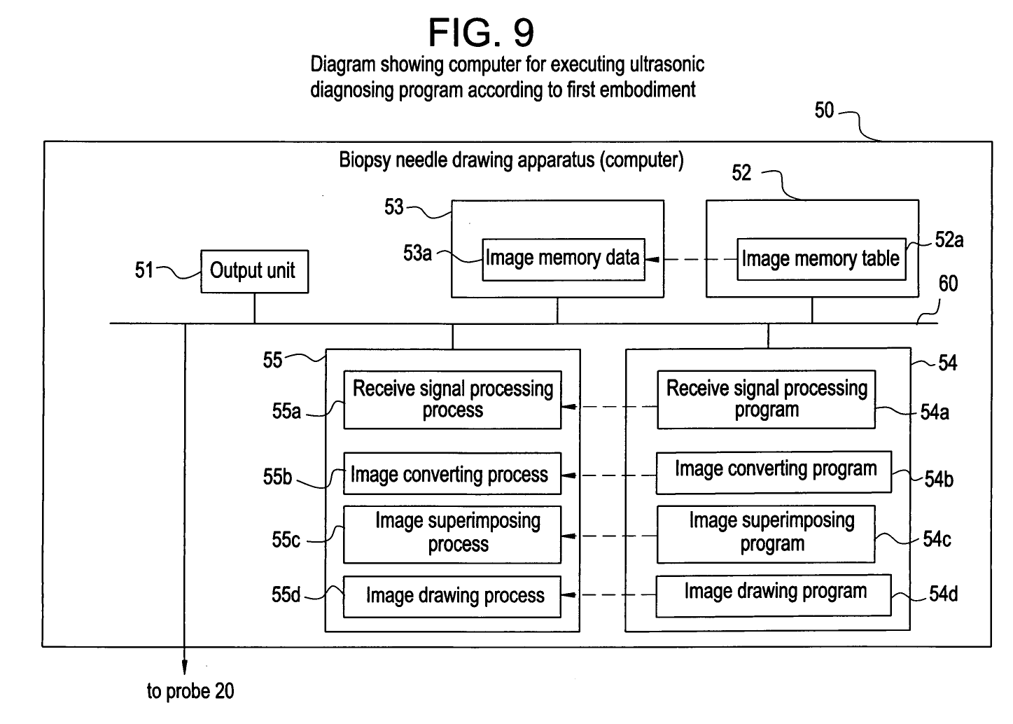

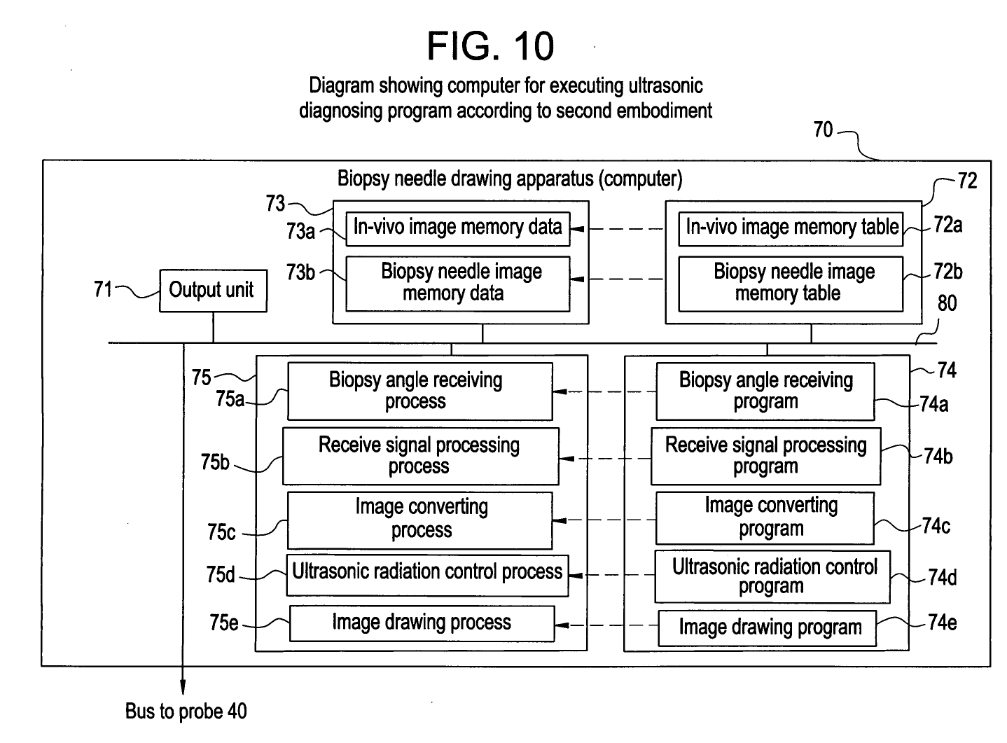

Meanwhile, the various processes described in the first and second embodiments can be realized by executing the programs prepared in advance by means of computer systems such as a personal computer, a work station, etc. Therefore, one example of a computer for executing a biopsy needle generating program having functions similar to each of the first and second embodiments, will be explained below using each of FIGS. 9 and 10. FIG. 9 is a diagram showing the computer for executing a biopsy needle generating program according to the first embodiment, and FIG. 10 is a diagram showing the computer for executing a biopsy needle generating program according to the second embodiment, respectively.

As shown in FIG. 9, the computer 50 used as an ultrasonic diagnostic apparatus comprises an input unit 51, an output unit 52, an HDD 53, a RAM 54, a ROM 55 and a CPU 56 all connected to one another by a bus 60. Here, the input unit 51 corresponds to the input unit 11 shown in FIG. 2, and the output unit 52 similarly corresponds to the output unit 12.

A biopsy needle generating program for exerting or exercising functions similar to the ultrasonic diagnostic apparatus 10 shown in the first embodiment, i.e., a receive signal processing program 55a, an image converting program 55b, an image superimposing program 55c and an image generating program 55d are stored in the ROM 55 in advance. Incidentally, these programs 55a, 55b, 55c and 55d may suitably be integrated or dispersed in a manner similar to the respective constituent elements of the ultrasonic diagnostic apparatus 10 shown in FIG. 2.

The CPU 54 reads these programs 54a, 54b, 54c, and 54d from the ROM 54 and executes them. Consequentially, as shown in FIG. 9, the respective programs 54a, 54b, 54c, and 54d function as a receive signal processing process 55a, an image converting process 55b, an image superimposing process 55c and an image generating process 55d respectively. Incidentally, the respective processes 55a, 55b, 55c, and 55d correspond to the receive signal processor 14a, the image converter 14b, the image superimposer 14c and the image generator 14d shown in FIG. 2, respectively.

As shown in FIG. 9, the HDD 53 is provided with an image memory stable 53a. The image memory table 53a corresponds to the image storage part 13a shown in FIG. 2. And the CPU 56 reads image memory data 54a from the image memory table 53a and stores it in the RAM 54, and executes a biopsy needle generating process on the basis of the image memory data 54a stored in the RAM 54.

Incidentally, the respective programs 55a, 55b, 55c and 55d need not necessarily be stored in the ROM 55 from the beginning. The respective programs are stored in, for example, “portable physical mediums” such as a flexible disk (FD), a CD-ROM, an MO disk, a DVD disk, a magneto-optical disk, an IC card, etc. inserted in the computer 50, or “fixing physical mediums” such as HDDs provided inside and outside the computer 50, and further “other computer (or server)” connected to the computer 50 via a public circuit, Internet, a LAN, a WAN, and the like, etc., and the computer 50 may read the respective programs from these and execute the same.

As shown in FIG. 10, the computer 70 used as an ultrasonic diagnostic apparatus comprises an output unit 71, an HDD 72, a RAM 73, a ROM 74 and a CPU 75 all connected to one another by a bus 80. Here, the output unit 71 corresponds to the output unit 31 shown in FIG. 6.

A biopsy needle generating program for exerting or exercising functions similar to the ultrasonic diagnostic apparatus 30 shown in the second embodiment, i.e., a biopsy angle receiving program 74a, a receive signal processing program 74b, an image converting program 74c, an ultrasonic radiation control program 74d and an image generating program 74e are stored in the ROM 74 in advance. Incidentally, these programs 74a, 74b, 74c, 74d and 74e may suitably be integrated or dispersed in a manner similar to the respective constituent elements of the ultrasonic diagnostic apparatus 30 shown in FIG. 6.

The CPU 75 reads these programs 74a, 74b, 74c, 74d and 74e from the ROM 74 and executes them. Consequentially, as shown in FIG. 10, the respective programs 74a, 74b, 74c, 74d and 74e function as a biopsy angle receiving process 75a, a receive signal processing process 75b, an image converting process 75c, an ultrasonic radiation control program 75d and an image generating process 75e respectively. Incidentally, the respective processes 75a, 75b, 75c, 75d and 75e correspond to the biopsy angle reception part 33a, the receive signal processor 33b, the image converter 33c, the ultrasonic radiation control part 33d and the image generator 33e shown in FIG. 6, respectively.

As shown in FIG. 10, the HDD 72 is provided with an in-vivo image memory table 72a and a biopsy needle image memory table 72b. The in-vivo image memory table 72a and the biopsy needle image memory table 72b respectively correspond to the in-vivo image storage part 32a and the biopsy needle image storage part 32b shown in FIG. 6. And the CPU 75 reads in-vivo image memory data 73a and biopsy needle image memory data 73b from the in-vivo image memory table 72a and the biopsy needle image memory table 72b and stores them in the RAM 73, and executes a biopsy needle generating process on the basis of the in-vivo image memory data 73a and the biopsy needle image memory data 73b stored in the RAM 73.

Incidentally, the respective programs 74a, 74b, 74c, 74d and 74e need not necessarily be stored in the ROM 74 from the beginning. The respective programs are stored in, for example, “portable physical mediums” such as a flexible disk (FD), a CD-ROM, an MO disk, a DVD disk, a magneto-optical disk, an IC card, etc. inserted in the computer 70, or “fixing physical mediums” such as HDDs provided inside and outside the computer 70, and further “other computer (or server)” connected to the computer 70 via a public circuit, Internet, a LAN, a WAN, and the like, etc., and the computer 70 may read the respective programs from these and execute the same.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus which applies ultrasound to within a body to be examined through an ultrasound probe, receives ultrasonic sounds reflected from within the body and a biopsy needle inserted in the body by means of the ultrasound probe and sequentially generates ultrasonic images, comprising:
an ultrasonic image storing device which sequentially stores the ultrasonic images therein;
an ultrasonic image superimposing device which sequentially superimposes the ultrasonic images stored in the ultrasonic image storing device in order of generation thereof; and
a superimposed-image generating device which generates an image superimposed by the ultrasonic image superimposing device.

2. The ultrasonic diagnostic apparatus according to claim 1, wherein the superimposed-image generating device superimposes images around the biopsy needle on one another.

3. The ultrasonic diagnostic apparatus according to claim 1, wherein the superimposed-image generating device adds the ultrasonic images stored in the ultrasonic image storing device every pixels.

4. The ultrasonic diagnostic apparatus according to claim 1, wherein the superimposed-image generating device superimposes the ultrasonic images stored in the ultrasonic image storing device on one another with being averaged out every pixels.

5. The ultrasonic diagnostic apparatus according to claim 1, wherein the superimposed-image generating device extracts the ultrasonic images highest in luminance on every pixel from the ultrasonic images stored in the ultrasonic image storing device.

6. The ultrasonic diagnostic apparatus according to claim 1, further comprising a setting changing device which accepts a predetermined change in setting from a user, wherein the superimposed-image generating device superimposes ultrasonic images on one another on the basis of the settings changed by the setting changing device.

7. An ultrasonic diagnostic apparatus which applies ultrasound to within a body to be examined through an ultrasound probe, receives ultrasonic sounds reflected from within the body and a biopsy needle inserted in the body by means of the ultrasound probe and sequentially generates ultrasonic images, comprising:
an ultrasonic radiation control device which applies the ultrasound vertically to the biopsy needle being inserted in the body;
an image converting device which converts a reflected signal of the biopsy needle obtained by the ultrasonic radiation control device to an image; and
an image generating device which generates an image corresponding to the biopsy needle imaged by the image converting device.

8. The ultrasonic diagnostic apparatus according to claim 7, wherein the ultrasonic radiation control device applies the ultrasound vertically to a target line displayed on the corresponding ultrasonic image by mounting an attachment to the ultrasound probe.

9. The ultrasonic diagnostic apparatus according to claim 7, further comprising a position detecting device which specifies a position of the biopsy needle, wherein the ultrasonic radiation control device applies the ultrasound vertically to the position specified by the position detecting device.

10. The ultrasonic diagnostic apparatus according to claim 7, wherein the ultrasonic radiation control device applies the ultrasound in plural directions while the biopsy needle is being inserted in the body and further applies the ultrasound in the direction in which a strong reflected signal is obtained.

11. The ultrasonic diagnostic apparatus according to claim 7, further comprising a setting changing device which accepts a predetermined change in setting from a user, wherein the ultrasonic radiation control device applies the ultrasound on the basis of the settings changed by the setting changing device.

12. The ultrasonic diagnostic apparatus according to claim 7, wherein the ultrasonic radiation control device further includes a combination generating device which applies the ultrasound vertically to the biopsy needle to generate image data of the biopsy needle and applies the ultrasound to the body to generate image data in the body, and which combines the image data in the body and the image data of the biopsy needle to generate an image.

13. The ultrasonic diagnostic apparatus according to claim 1, wherein each of the ultrasonic images is a two-dimensional or three-dimensional ultrasonic image.

14. An ultrasonic image generating method for applying ultrasound to within a body to be examined through an ultrasound probe, receiving ultrasonic sounds reflected from within the body and a biopsy needle inserted in the body by means of the ultrasound probe and sequentially generating ultrasonic images, comprising the steps of:
sequentially storing the ultrasonic images;
sequentially superimposing the stored ultrasonic images in order of generation thereof; and
generating the superimposed image.

15. An ultrasonic image generating method for applying ultrasound to within a body to be examined through an ultrasound probe, receiving ultrasonic sounds reflected from within the body and a biopsy needle inserted in the body by means of the ultrasound probe and sequentially generating ultrasonic images, comprising the steps of:
applying the ultrasound vertically to the biopsy needle inserted in the body;
converting a reflected signal of the biopsy needle obtained by ultrasonic radiation control device to an image; and
generating an image corresponding to the biopsy needle imaged by image converting device.

16. The ultrasonic diagnostic apparatus according to claim 7, wherein each of the ultrasonic images is a two-dimensional or three-dimensional ultrasonic image.
